(19) United States
(12) Patent Application Publication
(10) Pub. No.: US 2017/0258637 A1
(43) Pub. Date: Sep. 14, 2017

(54) DEVICE AND METHOD FOR PRODUCING PAINT MARKS ON AN EYE

(71) Applicant: Novartis AG, Basel (CH)

(72) Inventors: Theo Seiler, Erlangen (DE); Peter Riedel, Erlangen (DE); Christian Wuellner, Erlangen (DE)

(73) Assignee: Novartis AG, Basel (CH)

(21) Appl. No.: 15/108,538

(22) PCT Filed: Oct. 29, 2015
(86) PCT No.: PCT/EP2015/075147

§ 371 Date: Jun. 27, 2016

(51) Int. Cl. A61F 9/013; A61F 9/008; A61F 9/00; A61B 90/00

(52) U.S. Cl. CPC A61F 9/0136; A61B 90/39; A61F 9/00836; A61F 9/0026; A61B 2090/3933; A61B 2090/395; A61F 2009/00897; A61F 2009/00844; A61F 2009/00872

(57) ABSTRACT

A marker device for producing paint marks on an eye is disclosed. The marker device comprises a handling unit and a marker unit coupled to the handling unit for rotation with respect to the handling unit about a rotational axis. The marker unit includes at least two spaced apart marking surfaces and has a rotationally symmetric distribution of weight with respect to the rotational axis. A coupling between the handling unit and the marker unit permits the marker unit to rotate, due to gravity, relative to the handling unit into an equilibrium position.

### TECHNICAL BACKGROUND

In laser-assisted ophthalmic surgery, the refractive properties of an eye of a patient may be altered, for the purpose of correcting vision defects (e.g. hyperopia, myopia, astigmatism), by removal of corneal tissue with the aim to reshape the anterior surface of the cornea of the eye. One method to remove corneal tissue is by laser ablation, another method is by cutting out a volume of corneal tissue from the cornea using a cutting laser. Laser ablation is used, for example, in a procedure known as laser in-situ keratomileusis (LASIK). A cutting laser is used, for example, in a procedure named intracorneal lenticule extraction. In this procedure, a lens-shaped volume of corneal tissue (i.e. a lenticule) is cut away from surrounding tissue within the cornea. The lenticule is then removed through a channel leading to the outer surface of the eye.

Conventional ablating laser systems have a built-in eye-tracker to track eye motion during ablation and align the laser beam with the pupil center or another reference feature of the eye. Some eye-trackers offer a 6-dimensional tracking capability, i.e. they can track movements of the eye not only in three mutually orthogonal translational directions but additionally movements about three mutually orthogonal rotational axes. Conventional cutting laser systems used in ophthalmic surgery, conversely, require that the eye be fixated with respect to the laser system during laser operation using a suitable interface device (patient adapter), so that eye movement cannot occur during the laser treatment. It is for this reason that conventional cutting laser systems typically have no eye-tracking functionality.

An exemplary conventional patient adapter includes a suction ring and a coupling cone. The suction ring is placed onto the eye and fixed thereto by suction force. The coupling cone is mounted to the cutting laser system and has a contact member providing a contact surface for the eye. After the suction ring has been placed onto the eye, the coupling cone is lowered into the suction ring until the contact surface comes into contact with the outer surface of the eye. Depending on the shape of the contact surface, the eye may be deformed (e.g., levelled in the case of a plane contact surface) as a result of the contact surface being pushed against the eye.

Before the suction ring is placed onto the eye, the patient may be asked to stare with his eye to be treated at a fixed (e.g., red or green) light point created by the laser system. This light point serves as a means to ensure proper alignment of the visual axis of the eye with the laser system. The cutting pattern to be created in the eye is usually determined/calculated with respect to the pupil center as a reference point. But as soon as the suction ring has been placed onto the eye and the coupling cone has been coupled with the suction ring, the position of the eye's pupil center with respect to the laser system is invariably fixed. It is therefore important for the surgeon to position the suction ring on the eye as precisely as possible.

### SUMMARY OF THE DISCLOSURE

Conventional cutting laser systems offer the possibility for the surgeon to watch through an operating microscope the eye after its coupling to the laser system and during treatment. With some of these systems it is possible to shift the cutting pattern laterally in a coordinate system of the laser system with respect to the eye (i.e. in a plane orthogonal to the direction of beam emission from the laser system), based on the surgeon's observation of the eye through the operating microscope. For example, if the surgeon finds from his observation of the eye that the pupil is not properly centered with respect to the center of the suction ring (or with respect to a pair of cross hairs overlaid on the image of the eye that is displayed to the surgeon), he may manually steer the cutting pattern laterally until it has a desired position with respect to the pupil of the patient's eye.

The cutting pattern is oftentimes determined based on diagnostic data acquired in an upright position of the head of patient (e.g., while the patient is sitting on a chair). The laser treatment itself, conversely, is usually performed with the patient lying on a bed. It has been observed that the eye of a patient may undergo torsional movement about an axis intersecting the pupil during a change of the patient from the upright position to a lying position. For a cutting pattern that is rotationally symmetrical such torsion of the eye may be irrelevant. However, where the cutting pattern is rotationally non-symmetrical, it may be necessary to torsionally adjust the cutting pattern before the laser treatment is started.

Torsion compensation may generally include identifying a location of one or more features of an eye of a patient, these features including at least one of an anatomical structure of the eye (e.g. a blood vessel, an iris structure, etc.) and a paint mark applied to the eye.

One object of embodiments of the present invention is to provide a marker device that offers good positional reproducibility for applying paint marks on an eye.

In one aspect, embodiments of the present invention provide a marker device for producing paint marks on an eye. The marker device comprises a handling unit and a marker unit coupled to the handling unit for rotation with respect to the handling unit about a rotational axis. The marker unit includes a plurality of at least two spaced apart marking surfaces and has a rotationally asymmetric distribution of weight with respect to the rotational axis. The coupling between the handling unit and the marker unit permits the marker unit to rotate, due to gravity, relative to the handling unit into an equilibrium position.

According to certain embodiments, the marker unit includes at least one pair of marking surfaces disposed diametrically opposite to each other with respect to the rotational axis. In these embodiments, a pair of marking surfaces may be arranged opposite each other along a horizontal line intersecting the rotational axis when the marker unit is in the equilibrium position. Alternatively or additionally, a pair of marking surfaces may be arranged opposite each other along a vertical line intersecting the rotational axis when the marker unit is in the equilibrium position.

According to certain embodiments, the marker unit includes at least three, and for example a total of four, marking surfaces disposed at equal angular distances about the rotational axis.

According to certain embodiments, at least one, and for example each, of the plurality of marking surfaces has a concave shape. The concave shape may be selected to substantially match with the curvature of the outer surface of a human eye.

According to certain embodiments, the marker unit includes a main body and a plurality of marking protrusions projecting from the main body, wherein a marking surface is disposed at each of said marking protrusions.

Each of the marking protrusions may have a shape of a rib, the rib extending in a rib longitudinal direction and a having a breast surface forming one of the marking surfaces. The rib longitudinal direction of each of the marking protrusions may intersect with the rotational axis. Each of the marking protrusions may have a length from about 2 mm to about 4 mm in the rib longitudinal direction in the region of the breast surface.

According to certain embodiments, the main body has a design of an annular body.

According to certain embodiments, a distance between an edge of each marking surface, the edge facing the rotational axis, and the rotational axis is from about 3 mm to about 4.5 mm or from about 3.5 mm to about 4 mm when viewed in a plane orthogonal to the rotational axis.

According to certain embodiments, the handling unit includes a handling rod which, when viewed in a plane orthogonal to the rotational axis, projects radially from the marker unit. The handling rod may have a rod longitudinal direction extending at an acute angle of at most 30 degrees or at most 20 degrees or at most 10 degrees with respect to a plane orthogonal to the rotational axis. A length of the handling rod may be at least the size of a human palm.

According to another aspect, embodiments of the present invention provide a method for producing paint marks on an eye, the method comprising: providing a marker device as defined above; applying paint to one or more of the plurality of marking surfaces of the marker device; holding the marker device in a spatial orientation permitting the marker unit to rotate, due to gravity, relative to the handling unit into an equilibrium position; and contacting the eye with the marking surfaces in the equilibrium position of the marker unit to thereby transfer paint from the marking device to the eye.

### BRIEF DESCRIPTION OF DRAWINGS

Further elements, aspects and objects of the present invention may be taken from the subsequent discussion of specific embodiments shown in the accompanying drawings, in which:

FIG. 1 schematically shows a marker device according to an embodiment in a front view;

FIGS. 1a and 1b schematically show two practical realizations of the marker device of FIG. 1;

FIG. 2a schematically shows the marker device according to the embodiment of FIG. 1 in a side view;

FIG. 2b schematically shows the marker device of FIG. 2 in a situation of transferring paint from the marker device to an eye;

FIG. 3 schematically shows elements of a cutting laser system according to an embodiment;

FIG. 4 depicts images of an eye having exemplary paint marks, wherein the images correspond to an upright position and a lying position, respectively, of the eye;

FIGS. 5 to 7 are exemplary images of an eye having paint marks applied thereto, wherein one or more additional display symbols overlaid by a laser system are shown in the images.

### DETAILED DESCRIPTION OF DRAWINGS

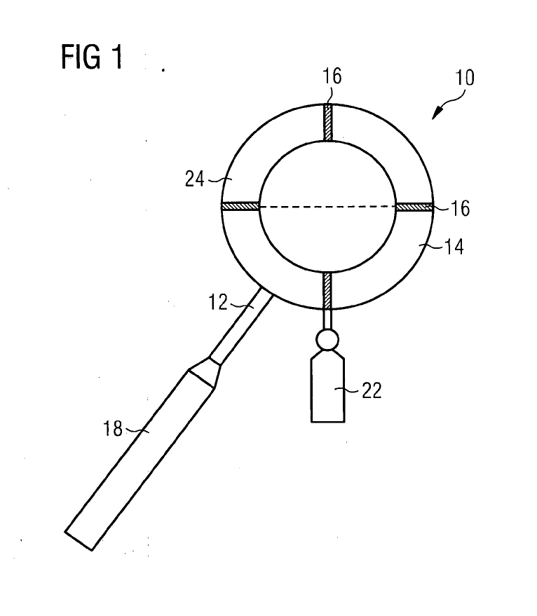

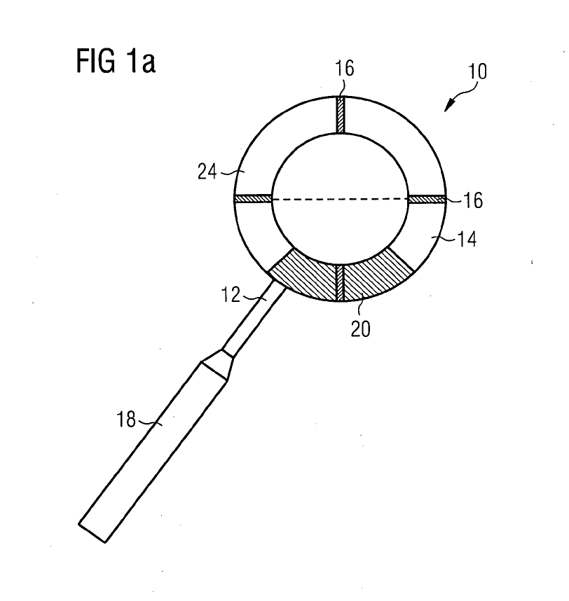

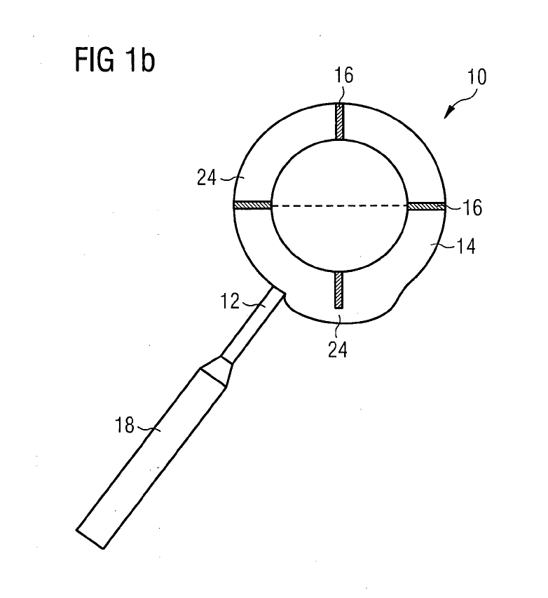

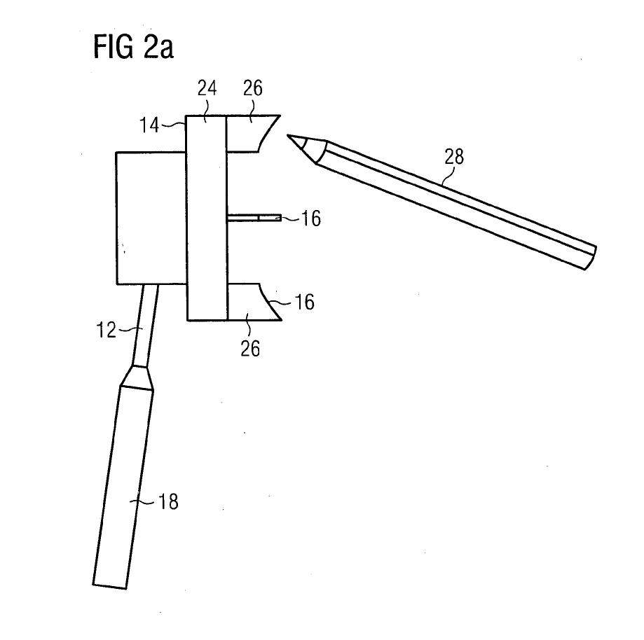

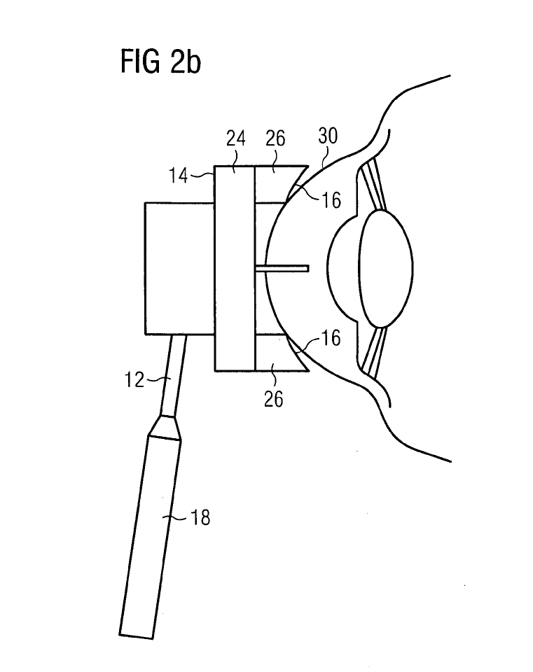

Reference is initially made to FIG. 1 and FIG. 2a. The marker device shown in these Figures is generally designated 10 and is useful for producing a plurality (in the exemplary case considered here: four) paint marks on the outer surface of an eye. The marker device 10 comprises a handling unit 12 and a marker unit 14. The marker unit 14 is coupled to the handling unit 12 such that the marker unit 14 can rotate with respect to the handling unit 12 about a rotational axis. The marker unit 14 includes four marking surfaces 16. The handling unit 12 of the marker device 10 includes a handling rod 18. The handling rod 18 is formed such that a user (for example a surgeon, an ophthalmologist or any other person) may conveniently grab the marker device 10 and hold it in either its right or its left hand while using it. As shown in FIG. 1, the handling rod 18 projects radially from the marker unit 14 when viewed in a plane orthogonal to the rotational axis. This ensures that the handling rod 18 and subsequently the hand of the user are not in line of sight with the marking unit 14, serving for better operability of the marker device 10.

The handling unit 12 may be formed from metal or from plastic or any other suitable material. It is possible that the handling unit 12 is integrally formed from a single material. Alternatively, the handling rod 18 may be formed from a different material than the rest of the handling unit 12. According to the examples shown in FIG. 1 and FIG. 2a, the handling rod 18 has a riffled surface to improve haptics, especially in order to prevent the marker device 10 from slipping out of the hand when in use.

Referring to FIG. 2a, the handling rod 18 extends longitudinally at an acute angle with respect to a plane orthogonal to the rotational axis. This structural feature may additionally improve user convenience since the user that holds the marker device 10 can comfortably position its hand during operation. The handling rod 18 may correspond in length approximately to the size of a human palm so that a user can properly encompass the handling rod 18 with its entire hand. Different lengths of the handling rod are of course possible in different embodiments of the invention.

As indicated in FIG. 1, the marker unit 14 has a rotationally asymmetric distribution of weight with respect to the rotational axis. Additionally, the marker unit 14 may have but is not limited to a rotationally symmetric design.

Referring to FIG. 1a, there is shown an embodiment of the present invention wherein the marker unit 14 has a rotationally symmetric design. The rotationally asymmetric distribution of weight is realized by manufacturing a predetermined area 20 of the marker unit 14 from a different material than the rest of the marker unit 14. The material from which the predetermined area 20 of the marker unit is manufactured has a higher mass per unit volume than the material from which the rest of the marker unit is formed. For example, the predetermined area 20 of the marker unit 14 may be manufactured from metal while the rest of the marker unit may be manufactured from plastic.

According to another embodiment of the invention as shown in FIG. 1b, the rotationally asymmetric distribution of weight of the marker unit 14 about the rotational axis is realized by providing the marker unit 14 with a rotationally asymmetric design. Therefore, additional material 24 is applied to a predetermined area of the marker unit 14. The additional material 24 may be a different material or the same material as used for manufacturing the rest of the marker unit 14. In either way, the additional material serves to shift the center of gravity of the marker unit 14 away from the rotational axis.

The rotationally asymmetric distribution of weight may additionally or alternatively be obtained by applying an additional weight 22 to the marker unit 14 by fixation means, as schematically shown in FIG. 1.

Both the embodiments in FIG. 1a and FIG. 1b respectively may but are not limited to have the same total weight. However, since the marker device 10 is handheld during use, the total weight of the device may not exceed ca. 50 g in certain embodiments in order to ensure convenient, yet precise handling of the marker device 10 by a user.

When the user holds the marker device 10 in one of his hands in a predetermined spatial orientation, the coupling between the handling unit 12 and the marker unit 14 permits the marker unit 14 to rotate, relative to the handling unit 12, into an equilibrium position. This rotation is merely induced by gravity. Besides the rotationally asymmetric distribution of weight of the marker unit 14, another prerequisite to provide this function is that the coupling is smooth. The rotationally asymmetric distribution of weight results in a torque when the marker unit 14 is in a position (hereinafter referred to as starting position) that does not correspond to the equilibrium position. For all possible starting positions of the marker unit 14, said torque has to overcome static friction of the coupling so that the marker unit 14 can move into the equilibrium position. The coupling may be continuously variable so that the static friction is independent from the starting position of the marker unit 14. The coupling may comprise but is not limited to a bearing such as an anti-friction bearing.

In the example embodiments shown in FIGS. 1 to 1b, the marker unit 14 includes a total of four marking surfaces 16. These marking surfaces 16 are disposed at equal angular distances about the rotational axis. When the marker unit 14 is in the equilibrium position, two of the marking surfaces 16 are disposed on a common horizontal line that intersects the rotational axis and two of the marking surfaces 16 are disposed on a common vertical line that intersects the rotational axis. Thus, the user of the marker device 10 may unambiguously produce the paint marks on an eye at exactly determined horizontal and vertical positions. The number of marking surfaces 16 is not limited to four. In other embodiments, the marker unit 14 may include two or three or more than four marking surfaces 16.

In an embodiment including two marking surfaces 16, the marking surfaces 16 may be disposed diametrically opposite to each other with respect to the rotational axis. The two marking surfaces 16 may be disposed on a common horizontal line that intersects the rotational axis when the marker unit 14 is in the equilibrium position.

In an embodiment including three marking surfaces 16, two of the marking surfaces 16 may be disposed diametrically opposite to each other with respect to the rotational axis. The third marking surface 16 may be disposed at an equal angular distance about the rotational axis with respect to the other two marking surfaces 16. The two of the marking surfaces 16 that are disposed diametrically opposite to each other may be disposed on a common horizontal line that intersects the rotational axis when the marker unit 14 is in the equilibrium position.

In an embodiment including more than four marking surfaces 16, the marking surfaces 16 may be disposed at equal angular distances about the rotational axis. Two of the marking surfaces 16 may be disposed on a common horizontal line that intersects the rotational axis when the marker unit 14 is in the equilibrium position. The more marking surfaces 16 the marker device 10 includes the more paint marks may be created on an eye. A higher amount of created paint marks may be advantageous for relocating the paint marks for torsion correction.

In the example shown in FIG. 2a, the marking surfaces 16 of the marker device 10 have a concave shape. The concave shape of the marking surfaces 16 may be adapted such that the marking surfaces 16 provide a match to the surface of a human eye. This makes the process of creating paint marks on an eye of a patient more comfortable for the patient. Nonetheless, the shape of the marking surfaces 16 is not limited to being concave. The marking surfaces 16 may also have the shape of a convex or a plane surface. The distance between an edge of each marking surface 16 and the rotational axis may be from about 3 mm to about 4.5 mm when viewed in a plane orthogonal to the rotational axis. Therein, the edge of each marking surface may face the rotational axis. The marker device 10 may thereby properly receive the eye such that the marking surfaces 16 only contact the eye in the peripheral area of the cornea. Thus, a central hole is left free in the middle, i.e. between the marking surfaces 16 and subsequently between the paint marks that are created on the eye. This prevents contact between the applied paint and pulsed laser beams during the removal of corneal tissue, since such contact would result in an at least partial absorption of the laser beams and subsequently negatively influence the process of removal of corneal tissue.

Referring again to the embodiment shown in FIG. 2a, the marker unit 14 includes a main body 24 and a plurality of marking protrusions 26 projecting from the main body. The number of marking protrusions 26 generally corresponds to the number of the marking surfaces 16 described above. Accordingly, one of the marking surfaces 16 is disposed at each of the marking protrusions. The marking protrusions 26 may have a projecting length of at least 3 mm, for example. As schematically illustrated in FIG. 2b, the projecting length should be sufficiently large to prevent the main body 24 from touching an eye when the marker device 10 is in contact with the eye. This may prevent an inadvertent injury to the eye during the creation of the paint marks.

Materials used for manufacturing the marker device 10 may be biocompatible and applicable in a surgical environment. The marking surfaces 16 may be manufactured from a flexible material to prevent the eye from being injured when paint marks are created. The marking surfaces 16 may further be adapted to easily absorb and release the paint applied thereto.

In the examples shown in FIGS. 1 to 2b, the marking protrusions 26 have a shape of a rib. It is noted that the shape of the marking protrusions 26 is not limited thereto. The marking protrusions 26 may for example have a shape of a T-beam or any other suitable shape. The rib extends in a rib longitudinal direction and has a breast surface forming one of the marking surfaces 16. The rib longitudinal direction of each of the marking protrusions 26 intersects with the rotational axis. The marking protrusions 26 may have a length from about 2 mm to about 4 mm in the rib longitudinal direction in the region of the breast surface. The marking protrusions 26 may additionally have a width of at least 0.2 mm. The created paint marks may thus exhibit good visibility for subsequently relocating the paint marks easily. A certain width may further be necessary to ensure precise torsion correction.

In the examples shown in FIGS. 1 to 2b, the main body 24 has an annular design. Additionally, the upper part of the handling unit 12 (i.e. in the area of the coupling between the handling unit 12 and the marker unit 14) has an annular design. Although the design of the main body 24 and the upper part of the handling unit 18 is not limited thereto, such design may provide the advantage that the user of the marker device 10 may see more of the patient's eye when creating the paint marks and may therefore position the marking device 10 more precisely. A further advantage arises from the fact that the user as well as the patient can look through the marker device 10 during creation of the paint marks. Thus, the process of creating the paint marks may be more convenient for the patient.

Referring now to FIG. 2a and FIG. 2b, the present invention provides a method for producing paint marks on a human eye. As a first step, the method includes providing a marker device 10 according to any of the embodiments described above.

A second step includes applying paint to two or more of the marking surfaces 16 of the marker device 10. As indicated in the example in FIG. 2a, this step may be carried out by painting the marking surfaces 16 with a suitable marker pen schematically illustrated at 28. Nonetheless, the method is not limited to a specific way for applying the paint. The paint may alternatively be applied by pressing the marking surfaces 16 into an ink pad. Another possibility to apply the paint may be to dip the marking surfaces 16 into liquid paint. Independent therefrom, the applied paint may be bio-compatible so that the patient is not exposed to any paint-related health risks.

A third step of the method includes holding the marker device 10 in a spatial orientation such that the marker unit 14 rotates, due to gravity, into an equilibrium position relative to the handling unit 12. It is not limiting for the functionality of the marker device 10 whether the user holds the marker device 10 in its left hand or in its right hand. Also, different users may hold the handling rod 18 of the marker device 10 at different positions within a plane transverse to the rotational axis in order to conveniently use the marker device 10. The equilibrium position may be reached when two of the marking surfaces 16 are disposed on a common horizontal line that intersects the rotational axis, as for example shown in FIG. 1. Alternatively, the equilibrium position may be reached when two of the marking surfaces 16 are disposed on a common vertical line that intersects the rotational axis. Therefore, the user that holds the marker device 10 may be able to unambiguously produce the paint marks on an eye at exact horizontal positions and/or exact vertical positions respectively.

As shown in FIG. 2b, a fourth step includes contacting an eye 30 of a patient with the marking surfaces 16. This step is conducted after the marker unit 14 has reached the equilibrium position. To ensure that the marker unit remains in the equilibrium position, the patient that receives paint marks on its eye has to be in an upright position, i.e. the patient is sitting or standing uprightly. Within this step, paint is transferred from the marking device 10 to the eye 30. The pressure applied to the eye 30 when creating the paint marks may be high enough to give the paint marks a good visibility but may be also low enough not to risk injuries of the corneal tissue of the patient.

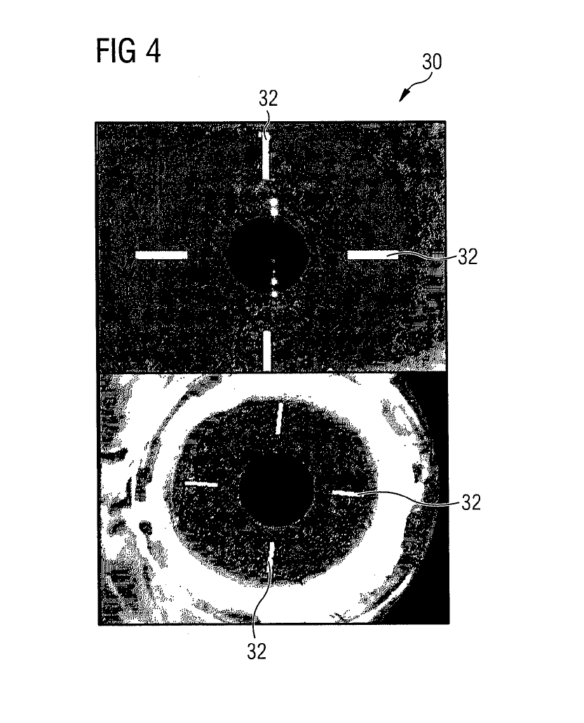

As shown in the example in FIG. 4, performing the four steps described above results in paint marks 32 that are created on the peripheral area of the cornea of the eye 30 of a patient. The paint marks 32 that are created when the patient is in an upright position are shown in the upper part of FIG. 4. The lower part of FIG. 4 shows the created paint marks 32 after the patient has moved into a lying position. Due to a motion of the eye, the paint marks 32 have rotated out of their exact horizontal and vertical positions.

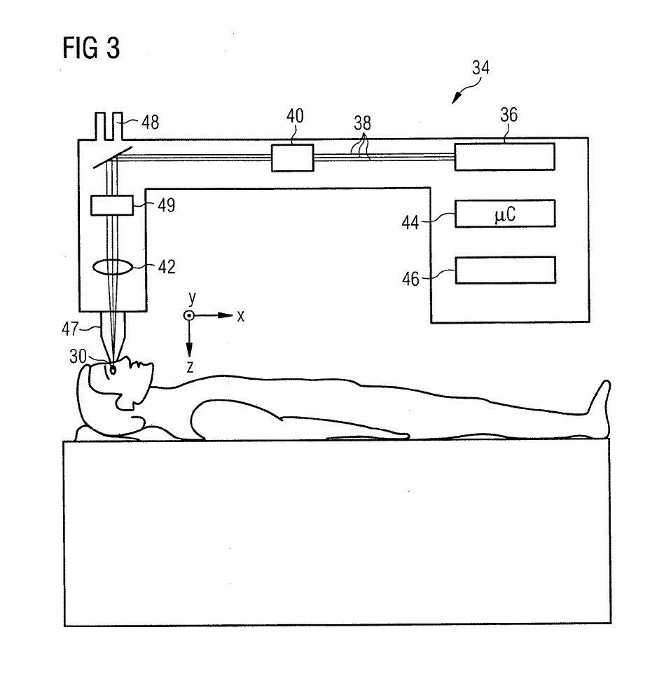

Referring now to FIG. 3, there is shown an example of a laser system 34 adapted to create one or more incisions (cuts) by laser radiation in an eye (such as, e.g., eye 30) of a patient resting in a lying position. The one or more cuts created by the laser system 34 may be required, e.g., to achieve a refractive correction of the eye 30. In certain embodiments, the laser system 34 can be controlled to create one or more cuts in a cornea of the eye. Corneal cuts may need to be created, for example, for an intracorneal lenticule extraction in order to cut away a lenticule of corneal tissue from surrounding tissue areas.

The laser system 34 comprises a laser source 36 configured to generate a laser beam 38 and further includes a xyz-scanning device 40 to deflect a focus of the laser beam 38 both in a direction of beam propagation and in a plane transverse to the direction of beam propagation. In line with usual notation in the art, the direction of beam propagation may be referred to as a z-direction and a plane orthogonal to the z-direction may be referred to as a xy-plane. The laser source 36 may produce pulsed laser radiation with a pulse duration in the nanosecond, picosecond, femtosecond or attosecond range. The laser radiation emitted from the laser system 34 has radiation properties that ensure the generation of a laser-induced optical breakdown (LIOB) in tissue of the eye following application of a single pulse or a group of pulses of the radiation. The laser beam 38 is focused by a focusing unit 42 and applied to the eye 30 of the patient. During surgery, a control unit 44 controls the laser source 36 and the scanning device 40 in accordance with a control program stored in a memory 46 of the laser system 34. The control program represents a cutting pattern which is to be realized in the eye through generation of a series of photodisruptions (each based on a LIOB). The cutting pattern is defined with regard to a xyz-coordinate system of the laser system 34 and is usually determined based on diagnostic data acquired while the patient is in an upright position. Since the patient's eye may undergo torsion when the patient changes his position from upright to lying, an angular correction (torsion correction) may have to be applied to the cutting pattern in the xyz-coordinate system of the laser system 34 before the laser treatment is started.

A patient interface 47 serves to couple the eye 30 to the laser system 34. The patient interface 47 may include, in a manner conventionally known per se, a suction ring to be placed onto the eye 30 and a coupling cone detachably mounted to a housing portion of the laser system 34. The coupling cone can be lowered into the suction ring and can be held together with the suction ring by suction force. The coupling cone is provided with a contact element having a contact surface for the eye 30. In a state in which the eye 30 is coupled to the laser system 34 via the patient interface, the anterior surface of the eye 30 is in contact with the contact surface of the contact element and may, for example, be flattened as a result of such contact.

The laser system 34 further comprises an operating microscope 48 and an overlay unit 49. The operating microscope 48 permits the surgeon to observe the eye through the focusing unit 42 when the eye is coupled to the laser system 34. An example image of the eye 30 is shown in the lower part of FIG. 4. It is clearly shown that the paint marks 32 that have been created on the eye 30 of the patient have rotated clockwise, resulting in a shift of the coordinates of the predetermined cutting pattern. To correctly apply the cutting pattern during surgery, this shift must be corrected for. The overlay unit 49 is adapted to overlay, under control of the control unit 44, one or more display symbols on the image of the eye as seen by the surgeon through the microscope 48.

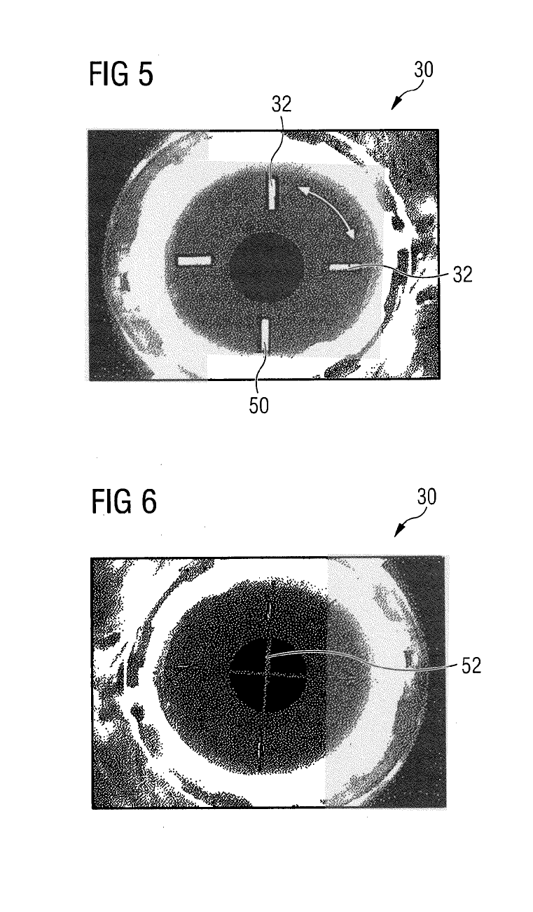

FIGS. 5 to 7 show examples on how the correction of the shift of coordinates in the predetermined cutting pattern may be performed. When paint marks 32 have been created on the eye 30 of the patient with the marker device 10 according to the present invention, the surgeon sees a live picture as shown in the lower part of FIG. 4 when looking through the operating microscope 48. To correct the shift of coordinates, the surgeon may exactly determine the amount of rotation of the eye 30.

Therefore, a second picture is superposed to the live picture by an overlay unit 49 of the laser system 34. The second picture contains elements (e.g. certain symbols) to simplify orientation for the correction process. It is therefore possible for the surgeon to readily identify a torsional movement of the eye 30 of a patient with respect to the xyz-plane of the laser system 34 that occurred when the patient moved into the lying position for surgery.

FIG. 5 shows four exact overlays 50 that are superposed to the four paint marks 32 in the live picture by the overlay unit 49 using a second picture. By operating means (e.g. a joystick or certain keys on a keyboard) of the laser system 34, the surgeon may rotate the superposed picture until the overlays 50 perfectly match the created paint marks 32. According to the example and to other certain embodiments, not just torsion of the eye 30 may be corrected but also the lateral position of the cutting profile may be exactly adjusted by laterally moving the superposed picture by said operating means. When the correct position of the overlays 50 and subsequently of the second picture that is superposed to the live picture by the overlay unit 49 has been reached, the surgeon may save the corresponding shift in coordinates in the memory 46 of the laser system 34. This may for example be carried out by pressing a certain button on a keyboard to confirm an exact match of the two pictures. Generally spoken, the shift in coordinates that corresponds to the shift of the superimposed second picture with respect to the xyz-plane of the laser system 34 may then be applied by the control unit 44 of the laser system 34 to correct the cutting pattern to be realized in the eye 30 of the patient in order to generate the photodisruptions at the exact positions that were determined during diagnostics.

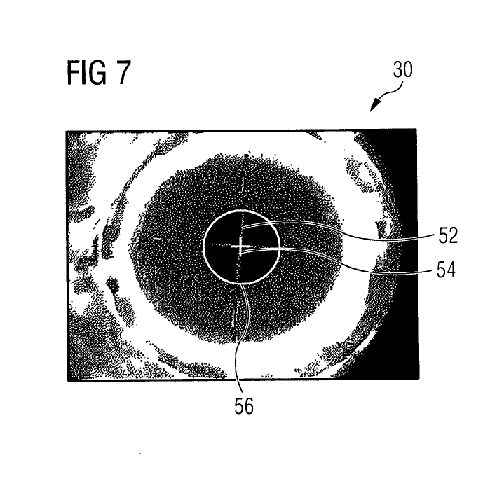

Alternatively and as shown in the examples in FIG. 6 and FIG. 7, a large crosshair 52 may be superposed to the live picture by the overlay unit 49 using a second picture. The width of the two legs of the crosshair 52 may correspond to the width of the created paint marks 32. By same operating means of the laser system 34 as discussed above, the surgeon may rotate the superposed picture until the crosshair 52 perfectly matches the created paint marks 32, as indicated in FIG. 6. In this way, precise torsion correction may be performed. For a lateral adjustment, the surgeon may refer to the intersection of the crosshair 52 and the pupil of the eye 30. The intersection of the crosshair 52 may be positioned at the center of the pupil. As shown in FIG. 7 and additionally to superposing the exact overlays 50 or the crosshair 52 by the overlay unit 49 using a second picture, the center 54 and the edge 56 of the pupil may be automatically detected and additionally superposed by the overlay unit 49 to the live picture. This may provide the surgeon with additional elements for orientation when performing torsion correction. As in the embodiment of FIG. 5, when the corresponding elements (e.g. the crosshair and the paint marks) in the two pictures perfectly match, the surgeon may save the corresponding shift in coordinates in the memory 46 of the laser system 34 by confirmation. The shift in coordinates may then be applied by the control unit 44 of the laser system 34 to correct the cutting pattern to be realized in the eye 30 of the patient in order to generate the photodisruptions at the exact positions that were determined during diagnostics.

In another embodiment of the invention, the control unit 44 may automatically detect the created paint marks 32 on the eye 30 of the patient by image processing means. Said image processing means may or may not include the overlay unit 49. The laser system 34 then provides the surgeon with a suggestion for the lateral correction and for torsion correction. The surgeon may adapt, by operating means, the suggested shift in coordinates and save the shift in coordinates in the memory 46 of the laser system 34 for a subsequent correction of the cutting pattern. Alternatively, it may be envisaged by the invention that the laser system directly applies the resulting shift in coordinates to the cutting pattern.

1. A marker device for producing paint marks on an eye, comprising:
a handling unit;
a marker unit coupled to the handling unit for rotation with respect to the handling unit about a rotational axis, the marker unit comprising a plurality of spaced apart marking surfaces and having a rotationally asymmetric distribution of weight with respect to the rotational axis; and
a coupling that couples the handling unit and the marker unit, the coupling permitting the marker unit to rotate, due to gravity, relative to the handling unit into an equilibrium position.

2. The marker device of claim 1, wherein at least one pair of marking surfaces are disposed diametrically opposite to each other with respect to the rotational axis.

3. The marker device of claim 1, wherein:
there are at least three marking surfaces; and
the marking surfaces are disposed at equal angular distances about the rotational axis.

4. The marker device of claim 1, wherein at least one of the marking surfaces has a concave shape.

5. The marker device of claim 1, wherein:
the marker unit comprises a main body and a plurality of marking protrusions projecting from the main body, and
a marking surface is disposed at each of the marking protrusions.

6. The marker device of claim 5, wherein each of the marking protrusions has a shape of a rib, the rib extending in a rib longitudinal direction and a having a breast surface forming one of the marking surfaces.

7. The marker device of claim 6, wherein the rib longitudinal direction of each of the marking protrusions intersects with the rotational axis.

8. The marker device of claim 6, wherein each of the marking protrusions has a length from about 2 mm to about 4 mm in the rib longitudinal direction in the region of the breast surface.

9. The marker device of claim 5, wherein the main body has a design of an annular body.

10. The marker device of claim 5, wherein a distance between an edge of each marking surface, the edge facing the rotational axis, and the rotational axis is from about 3 mm to about 4.5 mm when viewed in a plane orthogonal to the rotational axis.

11. The marker device of claim 1, wherein the handling unit includes a handling rod that, when viewed in a plane orthogonal to the rotational axis, projects radially from the marker unit.

12. The marker device of claim 11, wherein the handling rod has a rod longitudinal direction extending at an acute angle of at most 30 degrees with respect to a plane orthogonal to the rotational axis.

13. The marker device of claim 11, wherein a length of the handling rod is at least the size of a human palm.

14. A method for producing paint marks on an eye, the method comprising:
providing a marker device as defined in any one preceding claim;
applying paint to one or more of the plurality of marking surfaces of the marker device;
holding the marker device in a spatial orientation permitting the marker unit to rotate, due to gravity, relative to the handling unit into an equilibrium position; and
contacting the eye with the marking surfaces in the equilibrium position of the marker unit to thereby transfer paint from the marking device to the eye.
